(19) United States
(12) Patent Application Publication
(10) Pub. No.: US 2005/0202556 A1
(43) Pub. Date: Sep. 15, 2005

(54) Process and spotting solution for preparing microarrays

(76) Inventors: Walter Gumbrecht, Herzogenaurach (DE); Daniela Kuehn, Hemhofen (DE); Manfred Stanzel, Erlangen (DE)

Correspondence Address: HARNESS, DICKEY & PIERCE, P.L.C., P.O.BOX 8910, RESTON, VA, 20195, US

(21) Appl. No.: 11/023,372

(22) Filed: Dec. 29, 2004

(30) Foreign Application Priority Data
Dec. 29, 2003 (DE) 10361395.1

(51) Int. Cl. C12Q 1/68; C12M 1/34; B05D 3/00

(52) U.S. Cl. 435/287.2; 427/2.11

(57) ABSTRACT

A process is proposed for preparing a microarray including a multiplicity of analytical positions or spots and arranged on a support. The spots include probe molecules and a polymer which has been solidified to give a film. A spot is generated by applying an initially flowable spotting solution to the support, which solution includes a polymer and probe molecules, the polymer being solidified after application to the support. A spotting solution is further proposed which includes, in aqueous solution, probe molecules and a polymer which can be solidified in a non-free radical manner.

The present application hereby claims priority under 35 U.S.C. §119 on German patent application numbers DE 10361395.1 filed Dec. 29, 2003, the entire contents of which is hereby incorporated herein by reference.

### FIELD OF THE INVENTION

The present invention generally relates to a process for preparing microarrays of individual spots. In addition, the invention also generally relates to the corresponding spotting solution.

### BACKGROUND OF THE INVENTION

Biochips are increasingly used in biological analysis technology and medical technology. A biochip includes a usually planar support made of plastic or glass, on which a multiplicity of analytical positions or spots are arranged in the form of a microscreen or microarray. A spot contains capture or probe molecules. These are understood as meaning biochemical molecules or structures, for example DNA oligomers, proteins or haptens, to which biological molecular structures, for example in the form of DNA sequences, antibodies or enzymes, couple.

It is possible for a multiplicity of various analyses to be carried out in parallel side-by-side on a biochip, if the spots or groups of spots contain in each case different probe molecules which undergo pairing or hybridization with a very particular target molecule.

The spots are applied to the support using, for example, fine cannulas of glass or metal or needles. In order for the probe molecules to adhere to the support, the driver is usually functionalized beforehand, i.e. it is treated so as for its surface to have functional groups to which probe molecules can dock by way of appropriate coupling groups.

For example, a support surface is treated so as to have epoxy groups to which, for example, amino-functionalized DNA probe molecules couple. Both such coupling reactions and hybridization reactions require an aqueous medium.

For the latter reason, spots are applied in the form of aqueous solutions. A problem here is the fact that the aqueous portion of the solution evaporates very rapidly, owing to the small amount of liquid-spots are droplets of from about one hundred to a few hundred μm in diameter. Therefore, in order to prevent this, measures of controlling the humidity are required.

The immobilization of probe molecules on metal surfaces, for example on gold surfaces of microelectrodes using thiol-functionalized probe molecules, follows a similar pattern. Microelectrodes are useful for electrical read-out of an analytical result.

Disadvantageously, the electrode surfaces are occupied with probe molecules, thus impairing the efficacy of recording the analytical result. Another disadvantage of the biochips illustrated is the fact that the probe molecules can be immobilized only in a monomolecular layer on the support surface, i.e. that the number of the probe molecules immobilizable in one spot is limited.

In the case of biochips disclosed in U.S. Pat. No. 5,981,734 A, the probe molecules are bound to a polymer rather than the support surface, which polymer is present as a film on the support surface. To prepare such a biochip, first a large-area film is generated on the support surface by applying to the surface and then polymerizing a monomer solution. After polymerization, the film must be rinsed in order to remove reagents such as polymerization initiators etc., which may still be present. In a further step, spotting solutions containing various probe molecules are then applied to the film in the pattern of the subsequent microarray.

### SUMMARY OF THE INVENTION

It is an object of an embodiment of the invention to propose a process and a spotting solution which allow microarrays to be prepared in a simplified manner.

An object may be achieved by a preparation process, and/or by a corresponding spotting solution.

According to an embodiment of the invention, a spot may be generated by applying to a support an initially flowable spotting solution including a polymer and probe molecules, the polymer, after having been applied to the support, being solidified to give a film.

While in the process disclosed in U.S. Pat. No. 5,981,734 A the generation of a microarray requires four steps with in each case different operations, namely
two-dimensional application of a polymerizable substance mixture to a support,
polymerization of the substance mixture to give a film,
rinsing of the film and
application of spotting solutions in the pattern of the microarray to be generated,
   the process proposed according to an embodiment of the invention requires only two steps. A film-forming polymer is applied and the spots are generated in a first step essentially simultaneously and by using only a single operation, namely, for example, by way of dispensing via microcannulas. A second step includes only solidifying, for example crosslinking, the polymer, and this can be accomplished with little technical effort, for example by storing the supports, previously formulated in the manner described, in a humid environment.

In an embodiment of the invention, advantageously, the film can usually be solidified so gently that the probe molecules or the probe substance are not damaged in the process, as would be the case, for example, in a UV-induced free-radical polymerization. The process may be, for example, to dry the spotting solution in a purely physical manner, with the polymer being retained on the support in the form of a film in which the probe substance is embedded.

Another apparent advantage is the fact that it is possible to use an embodiment of the invention for preparing in a simple manner microarrays which are variable not only with respect to the probe molecules present therein but also with respect to their polymer film. This enables the polymer matrix to be adapted to the particular probe substance, for example with respect to coupling chemistry or to the type of immobilization of the probe molecules in the polymer matrix. In contrast, in the case of the biochips disclosed in U.S. Pat. No. 5,981,734 A, the same polymer matrix is present in all spots, i.e. the various probe molecules are always surrounded by the same polymer matrix. Moreover, the spots there are connected with one another via the polymer matrix, and this may result in contaminations and, during a measuring process or an analysis, in “chemical crosstalk”.

In a preferred variant of the process, the polymer is solidified by non-free radical crosslinking. In this connection, it is advantageously possible to add to the spotting solution a bifunctional crosslinker which couples, for example, to functional groups of the polymer by way of an addition reaction and which forms bridges between various polymer strands or different regions of a polymer strand.

In a preferred embodiment of the process of the invention, a polymer is intended to be used which has first and second radicals R1 and R2, in each case with linker groups. The linker groups are chosen here so as to connect to the respective other linker group, after application of the spotting solution and, where appropriate, after an activation, for example in the form of a temperature increase and, where appropriate, also an increase in humidity.

The consequence of the latter procedure is a crosslinking of various polymer strands or different regions of the same polymer via R1-R2 or R2-R1 bridges. Groups which are particularly suitable for this are epoxy and hydroxyl groups. Preference is given to using a polymer which contains 2-hydroxyethyl ester radicals as R1 and glycidyl ester radicals as R2. A polymer of this kind crosslinks even as a result of a small increase in temperature, for example to 50° C., and with relative humidities of about 70%, with the 2-hydroxy-ethyl ester radical of one polymer strand or strand section forming an addition compound with the glycidyl ester radical of another polymer strand or strand section. Likewise suitable are polymers in which R1 is a glycerol ester radical or an amide, for example an acrylamide.

Generally, probe molecules may be immobilized in the film in any manner. However, in a preferred variant of the process, the probe molecules are covalently bound to a radical of the polymer, after the spotting solution has been applied to a support. If probe molecules are used whose coupling group is an amino group, they can attach to the epoxy group. The radical R2 thus serves to simultaneously crosslink the polymer and to couple a probe molecule, i.e. it has dual function.

For an analysis carried out using a microchip, it is sufficient in principle for target molecules present in an analyte solution contacted with a microarray to react on the surface of a spot with probe molecules immobilized there. However, it is possible to increase the sensitivity or reaction rate of the biochip when using polymers which form hydrogels. Such a hydrogel is a loose polymer network which is entered readily by an analyte solution and target molecules present therein which find there a reaction space for the hybridization with probe molecules, which takes place in an aqueous environment. Preference is given to using copolymers of 2-hydroxyethyl methacrylate or glyceryl methacrylate and glycidyl methacrylate. The ester radicals of the copolymers ensure high swellability in water and serve, at the same time, to crosslink and immobilize probe molecules.

In a further preferred variant of the process, a hydrophilic, nonvolatile filler, in particular a water-swellable polymeric filler, is intended to be added to the spotting solution, thereby diluting the latter. The result of this measure is a widening of the polymer network and thus an enlargement of the reaction space available within a spot. This offers the possibility of making accessible a larger number of probe molecules within a spot for target molecules and thus increasing the sensitivity of a biochip. Advantageously, the filler is removed again by washing after crosslinking of the polymer. However, this does not require any further processing step, rather the filler may be removed in the course of an analysis, when contacting a microarray with an aqueous analyte solution. A filler which meets the requirements mentioned particularly well is polyvinylpyrrolidone, a substance used as thickener in other technical fields.

The viscosity of the spotting solution, in particular when a filler of the type mentioned has been added, is most usually too high to be able to apply the solution to a support, for example with the aid of microcannulas or jets. In a further preferred variant of the process, therefore, the spotting solution is intended, prior to application to a support, to be admixed with a water-miscible solvent which reduces the viscosity of the solution and which evaporates rapidly at room temperature, i.e. whose vapor pressure is sufficiently high at room temperature. After evaporating the solvent at least partially, the spots have a consistency which allows both transport and relatively long storage, before the polymer is crosslinked.

The latter procedure of spot arrays prepared according to an embodiment of the invention enables, for example, an entire day's production to be initially stored in order to be subjected later to joint heat treatment to solidify or crosslink the polymer. This is preferably achieved by storage in a humid environment, for example at from 40 to 50° C. and a relative humidity of at least 50% for a period of from about 8 to 36 hours. The solvent used of the type discussed is a pyrrolidone derivative, preferably 1-methyl-2-pyrrolidone.

### BRIEF DESCRIPTION OF THE DRAWINGS

The invention will now be illustrated further by reference to the attached figures in which:

FIG. 1 to FIG. 3 depict diagrammatic representations of various variants of the process,

FIG. 4 depicts a diagrammatic representation which illustrates the preparation of a microarray in substeps A, B and C, and

FIG. 5 depicts a diagrammatic representation of an analytical application of a microarray.

### DETAILED DESCRIPTION OF THE EXEMPLARY EMBODIMENTS

It is intended to prepare an array of individual spots for a biochip. The individual spots are located on a support and contain capture molecules to which, in the subsequent biochemical analysis, molecules can dock according to the key/lock principle. Preparation of the spot array requires a suitable spotting solution. Furthermore required are technical dispensing devices/methods in order to place the spots on the base at a micrometer distance.

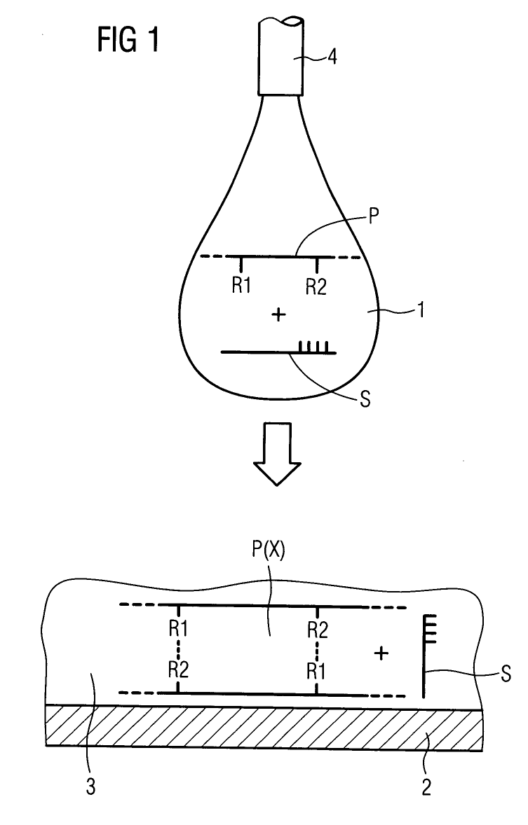

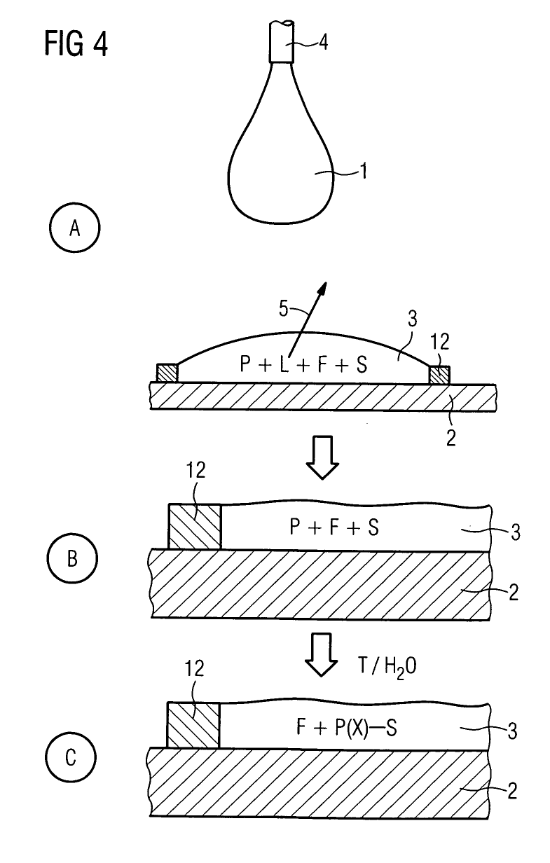

In the variant of the process, depicted in FIG. 1, the main components present—where appropriate, in addition to other additives, for example a filler and a volatile solvent—in the spotting solution 1 are a polymer P and a probe substance which includes probe molecules S, for example in the form of oligonucleotides. Very generally, the polymer P is a film-forming polymer and forms, after application to a support 2, in particular by way of dispensing using microcannulas 4, as depicted in FIG. 4A, a microdroplet or spot 3 which solidifies at room temperature or at slightly elevated temperatures.

The solidification of the polymer P to give a film may be carried out, for example, in a physical manner. However, preference is given to using a polymer P which can be covalently crosslinked in a non-free radical manner, with the crosslinked polymer P(X) forming a film or at least the structuring main component of such a film.

The variants of the process which are indicated in FIGS. 1 to 3 make use of a polymer P which has radicals R1 and R2 which can be covalently crosslinked with one another. Apart from, where appropriate, a crosslinking initiator, no further chemicals are required for the crosslinking. This is advantageous in that the risk of an impairment—for example a partial inactivation of the probe molecules S—is less likely, if fewer foreign substances are present in the spotting solution.

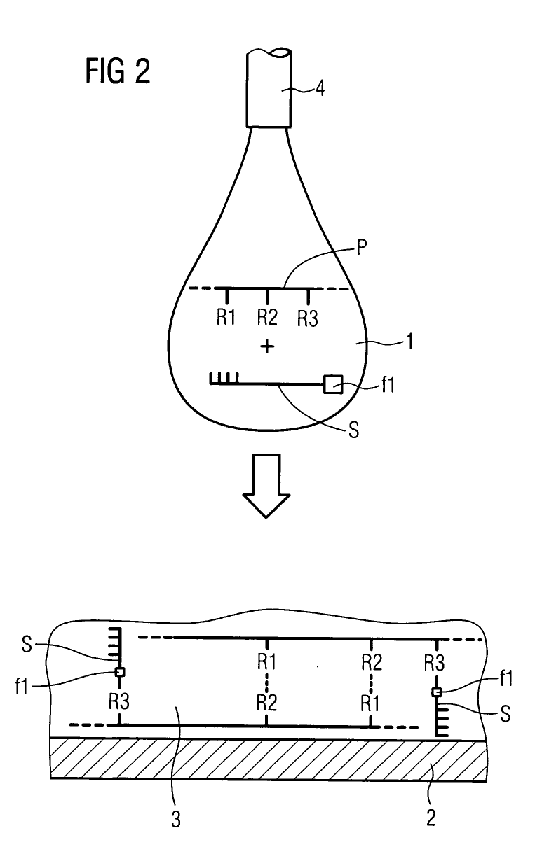

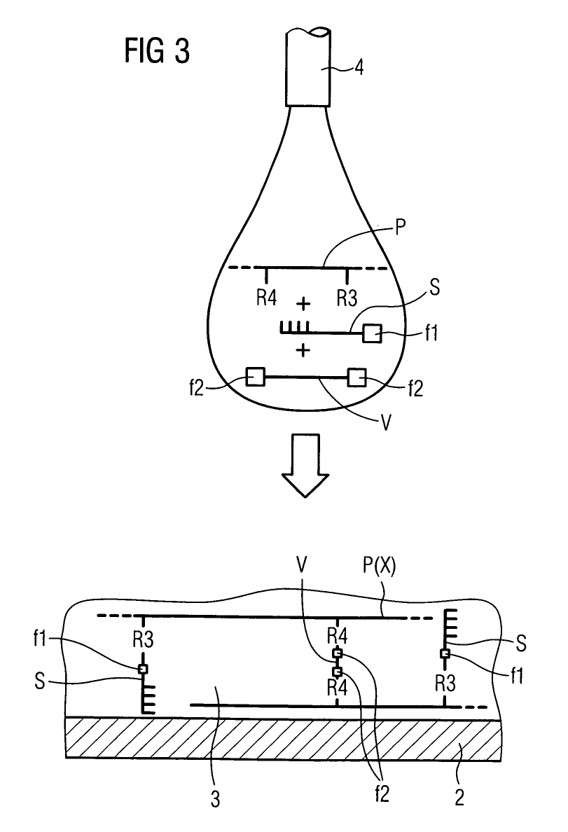

The probe molecules S may be immobilized in the spot 3 in principle in any manner, for example by way of hydrogen bridges. In this case, the probe molecules need not have any coupling groups (FIG. 1). However, in order to prevent, when an analyte solution 7 is applied to a spot 3, probe molecules S from entering the solution, preference is given to binding the probe molecules S covalently. The latter then have at one end a coupling group f1 which can link up to a radical R3 of the polymer P, as depicted in FIGS. 2 and 3.

Coupling between a probe molecule S and a radical R3 is carried out together with crosslinking of the polymer. However, alternatively it is also conceivable for coupling of the probe molecule S to a radical R3 of the polymer to be carried out already in the spotting solution or for a polymer to be used to which probe molecules have already bonded. After such a spotting solution 1 has been applied to a support 2, only crosslinking of the polymer P is still carried out.

The radical R3 may be different from R1 or R2. However, with respect to polymer preparation, for example, it may also be advantageous if a radical suitable for crosslinking the polymer simultaneously also serves to couple probe molecules S, i.e. if R3 is identical to R1 or R2 or at least functionally equivalent thereto.

The polymer may also be crosslinked, as depicted in FIG. 3, by a bivalent crosslinker V with terminal functional groups f2 which couple to a radical R4 of the polymer. In this case, it is also conceivable that R4 simultaneously serves to couple probe molecules. R4 could be, for example, a glycidyl ester radical to whose epoxy group amino-functionalized probe molecules couple. A suitable crosslinker V in this case would be, for example, a diol, i.e. a dihydric alcohol.

The consistency or, for example, polymer mesh size of a spot 3 decides whether or to what extent target molecules Z present in an analyte solution penetrate the spot 3. If a spot 3 is formed by a film having a relatively solid consistency, then a reaction between the probe molecules S and the target molecules Z is more likely to take place at the spot surface. To increase the sensitivity of a biochip it is then advantageous if as large a number as possible of hybridization reactions between probe molecules and target molecules take place per unit area of the spot. This is achieved if a spot has a relatively large volume and a consistency which allows analyte solution and target molecules to enter easily.

Such conditions are present in spots formed by polymeric hydrogels. Preference is given to using copolymers of 2-hydroxyethyl methacrylate and glycidyl methacrylate or of glyceryl methacrylate and glycidyl methacrylate. They form loose wide-mesh polymer networks and can be crosslinked to one another, without further radicals or functional groups having to be incorporated into the polymer for this purpose. The hydroxyl group of the 2-hydroxyethyl ester radical and the epoxy group of the glycidyl ester radical react with one another in a humid environment of from about 40 to 75° C., with preferably a relative humidity of at least 50% being maintained.

Probe molecules also couple to the polymers under such conditions. If the chosen coupling group f1 of a probe molecule S is an amino group, binding to the glycidyl ester radical of the polymethacrylates mentioned takes place. The number and distribution of the ester radicals are generally not strictly defined but may be varied in order to adapt to the particular analytical tasks and to the different conditions present in each case. It is also possible to incorporate further radicals into the polymers, for example in order to optimize the properties of a film or to bind probe molecules S.

When crosslinking a polymer in the manner described above, it may be possible for the polymer network produced to be too close-meshed, and this would result in a film having a relatively solid consistency.

A spot with such a film would not be swellable to the desired extent or would not provide an optimal reaction volume for the hybridization reaction between probe molecules S and target molecules Z, which takes place in an aqueous environment. This may be overcome if a hydrophilic polymeric filler F, in particular polyvinylpyrrolidone, is added to the spotting solution 1. Polyvinylpyrrolidone is used as thickener in other technical areas. The filler additive causes a reduction in the concentration of the polymer in the spotting solution, i.e. the polymer strands have a lower concentration, resulting in a loose wide-mesh polymer network in crosslinking. At the same time, a substance such as polyvinylpyrrolidone increases the viscosity of the spotting solution 3. In this connection, the consistency can be adjusted in such a way that spots 3 applied to a support have a strength which permits relatively long storage.

However, in order to facilitate application of the spotting solution to a support, in particular in the case of dispensing with the aid of microcannulas 4, in particular according to FIG. 4, or to make the application possible at all, the spotting solution is admixed with a volatile solvent L which dissolves the polymer, the probe molecules and the filler, i.e. especially the filler polyvinylpyrrolidone. Solvents having these properties are pyrrolidone derivatives, with 1-methyl-2-pyrrolidone being particularly suitable. Other suitable solvents are dimethylformamide (DMF) and dimethyl sulfoxide (DMSO).

The spotting solution 1 applied to a support 2 with the aid of a microcannula 4 first adopts a drop shape or convex shape (FIG. 4A). Positionally accurate dispensing is facilitated when vertically protruding ring structures 12 are present on the support surface. A spot 3 has a diameter of from about 100 μm to a few hundred μm. The solvent L evaporates into the environment within a few seconds to minutes, as indicated in FIG. 4A by the arrow 5. The spot 3 becomes flatter, with its volume or its thickness 6 being determined now mainly by the proportion of polymer and filler and being about 10 to 100% of the average spot diameter.

A first process step is completed after the at least partial evaporation of the solvent L.

The corresponding biochips or supports 2 are indicated in FIG. 4B and may either be processed further immediately or be intermediately stored for at least up to a few hours, until the final solidification of the polymer film is effected, in particular by way of crosslinking. The consistency of a spot 3 after the at least partial evaporation of the volatile solvent L is such that the spot can withstand a certain pressure load or other mechanical load. This becomes noticeable when supports 2 are arranged on a tape or are part of such a tape.

The spots 3 or microarrays may then—more or less in a conveyor belt process—be applied to the tape which is then, where appropriate with a spacer film as an intermediate layer, rolled up into a roll. Such a roll or several such rolls may then, where appropriate after intermediate storage, be subjected to a joint humidity-heat treatment in order to crosslink the polymer P and to immobilize probe molecules S on the polymer, according to FIG. 4C.

After crosslinking, a loose polymer network is present, caused inter alia by the presence of the filler F. The probe molecules S are bound covalently to this network. In the case of biochips which can be read out electrically and which have, for example, contamination-sensitive gold electrodes, the virtually gas-impermeable polymer film covers the microelectrodes of a spot and protects them from harmful environmental influences.

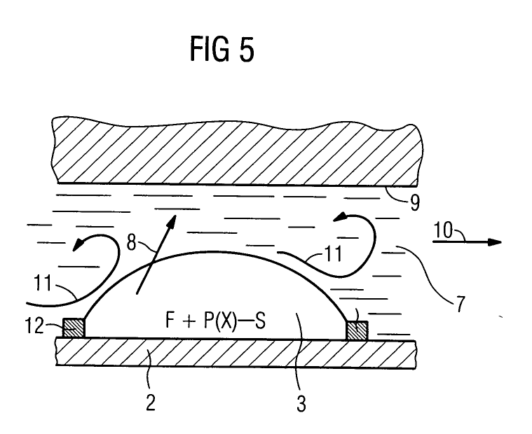

An analysis may be carried out as indicated in FIG. 5. If an analyte solution 7 containing target molecules Z is applied to a microchip or to a microarray of spots 3, the solution penetrates all spots 3 with a binding S-Z taking place only in spots with matching probe molecules S. A spot 3 which comes into contact with an aqueous analyte solution 7 or else with a different solution, for example a PCR product, adopts again, due to water uptake and swelling, a drop shape or convex shape. The swelling and volume enlargement is then caused primarily by the polymer hydrogel.

The filler F, which is e.g. polyvinylpyrrolidone, is virtually immediately removed, at least partially, from the spots 3 by the analyte solution 7, as indicated in FIG. 5 by the arrow 8. The spatial expansion of the spot 3 is then primarily caused by the polymer network and water embedded therein. The target molecules Z which have penetrated a spot 3 thus have available an aqueous reaction space which is free of filler F. The filler F may also be removed in a separate operation, for example by rinsing with water or a buffer.

Automated devices into which a biochip has been integrated or can be integrated are frequently used in analytical applications. According to FIG. 5, for example, the analyte solution 7 flows via a microchannel 9 over the biochip or the spots 3 arranged thereon. The direction of flow 10 of the analyte solution 7 here runs parallel to the flat plane of the support 2. An intensive mass transfer between spot 3 and analyte solution 7 is promoted by the spots protruding into the flow and causing the analyte solution to eddy, as indicated in FIG. 5 by the arrows 11.

To prepare a microarray of spots (“spot array”), a group of microcannulas 4 is used which are arranged in the pattern of the microarray to be generated. For this purpose, it is possible to use, for example, a process and a device as described in the German patent application file No. 103 61 399.4-52 “Verfahren und Vorrichtung zum Dispensieren von Flüssigkeiten im Mikroraster”, which has the same application priority, the entire contents of which are hereby incorporated herein by reference. Further, the entire contents of corresponding U.S. application entitled “METHOD AND APPARATUS FOR DISPENSING LIQUIDS IN A MICRO-GRID PATTERN”, and filed on the same date as the present application, are also incorporated herein by reference.

The different spotting solutions for individual spots of the spot array contain a common basic solution which contains, for example, 5% by weight of one of the abovementioned copolymers based on acrylamide, 20% by weight polyvinylpyrrolidone, 65% by weight 1-methyl-2-pyrrolidone and 10% by weight water. A basic solution of this kind is admixed with a probe substance assigned to a spot 3 or to a group of spots 3.

The afore-described process can be used, in particular, to prepare spot arrays suitable for use in biochips. A specific mixture of polymer substances can be predefined for the preparation.

It is moreover possible by controlled adjustment of the composition of the substance mixture to ensure that the reaction of capture DNA and target DNA takes place either only on the surface or at least only in the outer layer of the hydrogel spot, thus making possible a rapid and efficient hybridization.

A component a of the mixture for the reaction layer exhibits the structure: 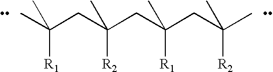
R1, R2 are the radicals of the polymer. Formulated as a polymethacrylic acid ester, the component a has the following structure:

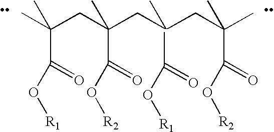

Specifically, the component a has the structure:

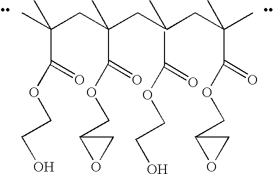

In the illustrations above, the distribution, i.e. the number and order, of R₁ and R₂ is not defined. Further components, but at least a third component R₃, may also be present.

When using a functionalized DNA capture oligonucleotide, the following applies:

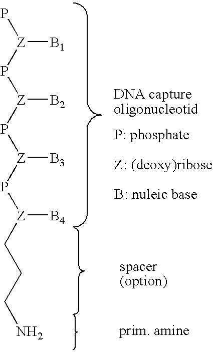

Unless an unreacted 2-hydroxyethyl ester radical (a) is present, DNA capture oligonucleotides b and c which are immobilized via a primary amine on the glycidyl ester radical result in a crosslinking (d,4 coupling) via 2-hydroxyethyl ester radical and glycidyl ester radical (hydroxylether), according to the following structural formula:

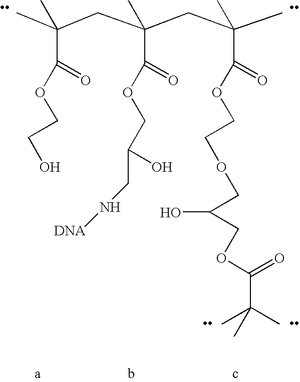

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A process for preparing a microarray, comprising:
arranging a multiplicity of analytic spots on a support, the spots including probe molecules and a polymer solidified to give a film, wherein a spot is generated by,
applying an initially flowable spotting solution to the support, the solution including a polymer and probe molecules, and
solidifying the polymer after it has been applied to the support.

2. The process as claimed in claim 1, wherein the polymer is solidified by non-free radical covalent crosslinking.

3. The process as claimed in claim 2, wherein crosslinking is carried out via radicals R1, R2 of the polymer, which have linker groups which are interconnectable to one another.

4. The process as claimed in claim 3, wherein a polymer is used whose radicals R1 include an epoxy group and whose radicals R2 include a hydroxyl group as linker group.

5. The process as claimed in claim 4, wherein R1 is a 2-hydroxyethyl ester radical and R2 is a glycidyl ester radical.

6. The process as claimed in claim 4, wherein R1 is a glycerol ester radical and R2 is a glycidyl ester radical.

7. The process as claimed in claim 1, wherein probe molecules are covalently bound to a radical of the polymer, after the spotting solution has been applied.

8. The process as claimed in claim 7, wherein probe molecules are used whose coupling group is an amino group.

9. The process as claimed in claim 1, wherein a polymer which forms a hydrogel is used.

10. The process as claimed in claim 9, wherein the polymer used is a copolymer of 2-hydroxyethyl methacrylate and glycidyl methacrylate.

11. The process as claimed in claim 9, wherein the polymer used is a copolymer of glyceryl methacrylate and glycidyl methacrylate.

12. The process as claimed in claim 1, wherein a hydrophilic, water-swellable or water-soluble nonvolatile filler is added to the spotting solution.

13. The process as claimed in claim 12, wherein a water-swellable polymeric filler is used.

14. The process as claimed in claim 13, wherein the filler is polyvinylpyrrolidone.

15. The process as claimed in claim 12, wherein, after crosslinking of the polymer, the filler is at least partially removed again from a spot by contacting the latter with water or an aqueous solution.

16. The process as claimed in claim 1, wherein the viscosity of the spotting solution is reduced, prior to application to a support, by adding a volatile solvent which is at least partially miscible with water and which evaporates again at least partially after said application.

17. The process as claimed in claim 12, wherein a solvent is used, in which the polymer, the probe molecules and the filler can be dissolved.

18. The process as claimed in claim 17, wherein the solvent used is a pyrrolidone derivative.

19. The process as claimed in claim 18, wherein the solvent is 1-methyl-2-pyrrolidone.

20. The process as claimed in claim 1, wherein the polymer is solidified and, where appropriate, the probe molecules are coupled to said polymer by subjecting the spots applied to a support to humid atmosphere.

21. The process as claimed in claim 20, further comprising:
treating the spots at a temperature of from 40 to 75° C. and a relative humidity of at least 50% for a period of from 8 to 36 hours.

22. A spotting solution for preparing microarrays according to a process as claimed in claim 1, wherein the solution includes a polymer which can be solidified in a non-free radical manner and probe molecules.

23. The spotting solution as claimed in claim 22, including a polymer having first and second radicals R1, R2 which have linker groups, interconnectable to one another.

24. The spotting solution as claimed in claim 23, wherein R1 includes an epoxy group and R2 includes a hydroxyl group as linker group.

25. The spotting solution as claimed in claim 24, wherein R1 is a 2-hydroxyethyl ester radical and R2 is a glycidyl ester radical.

26. The spotting solution as claimed in claim 24, wherein R1 is a glycerol ester radical and R2 is a glycidyl ester radical.

27. The spotting solution as claimed in claim 22, wherein the polymer has radicals for covalent binding of probe molecules.

28. The spotting solution as claimed in claim 27, wherein the coupling group present in the probe molecules is an amino group.

29. The spotting solution as claimed in claim 22, wherein the polymer is one which forms a hydrogel.

30. The spotting solution as claimed in claim 29, wherein the polymer is a copolymer of 2-hydroxyethyl methacrylate and glycidyl methacrylate.

31. The spotting solution as claimed in claim 29, wherein the polymer is a copolymer of glyceryl methacrylate and glycidyl methacrylate.

32. The spotting solution as claimed in claim 22, further comprising a hydrophilic, nonvolatile filler present therein.

33. The spotting solution as claimed in claim 32, wherein the filler is polyvinylpyrrolidone.

34. The spotting solution as claimed in claim 22, further comprising a viscosity-reducing, water-miscible solvent which is volatile at room temperature present therein.

35. The spotting solution as claimed in claim 34, further comprising a solvent which dissolves the polymer, the probe molecules and the filler present therein.

36. The spotting solution as claimed in claim 35, wherein the solvent present therein is a pyrrolidone derivative.

37. The spotting solution as claimed in claim 36, wherein 1-methyl-2-pyrrolidone is present therein.

38. The process as claimed in claim 13, wherein, after crosslinking of the polymer, the filler is at least partially removed again from a spot by contacting the latter with water or an aqueous solution.

39. The process as claimed in claim 14, wherein, after crosslinking of the polymer, the filler is at least partially removed again from a spot by contacting the latter with water or an aqueous solution.
